(19) United States
(12) Patent Application Publication
(10) Pub. No.: US 2025/0152294 A1
(43) Pub. Date: May 15, 2025

(54) TRACKING MOVING OBJECTS DURING THE AUTO-CONFIGURATION OF A SURGICAL MICROSCOPY SYSTEM

(71) Applicant: CARL ZEISS MEDITEC AG, Jena (DE)

(72) Inventors: Markus PHILIPP, Stuttgart (DE); Stefan SAUR, Aalen (DE); Neal BACHER, Stuttgart (DE)

(21) Appl. No.: 18/945,670

(22) Filed: Nov. 13, 2024

(30) Foreign Application Priority Data
Nov. 15, 2023 (DE) 10 2023 131 862.4

(51) Int. Cl. A61B 90/25; G02B 21/00; G02B 21/36; H04N 23/60; H04N 23/611; H04N 23/695

(52) U.S. Cl. CPC A61B 90/25; G02B 21/0012; G02B 21/365; H04N 23/611; H04N 23/64; H04N 23/695

(57) ABSTRACT

Techniques which relate to an automatic configuration of one or more components of a surgical microscopy system for imaging a moving object are disclosed. In the described techniques, the initially determined target configuration of the surgical microscopy system is selectively adapted if a movement of the moving object is detected; this selective adaptation takes place when the movement is within a predefined spatial limit and/or a predefined temporal limit.

### TECHNICAL FIELD

Various examples of the disclosure relate to techniques for automatically configuring a surgical microscopy system for imaging an object. Various examples of the disclosure relate to techniques for taking into account a movement of the object when controlling at least one component of the surgical microscopy system.

### BACKGROUND

Medical surgical microscopy systems (also referred to as robotic visualization systems or surgical visualisation systems) having a robotic stand for positioning a microscope are known from the prior art, see, for example, DE 10 2022 100 626 A1.

The robotic stand can be controlled manually. However, techniques are also known in which the robotic stand is controlled automatically, for example in order to enable auto-centring and/or auto-focusing on a particular object, for example a surgical instrument (also referred to as surgical equipment or a surgical tool). A user command triggers a positioning process during which the robotic stand and/or an objective optical unit of the microscope is/are controlled, i.e. actuated.

Such techniques for automatic control are known, for example, from U.S. Pat. No. 10,456,035 B2. An automatic position correction is described there. A deviation of the relative position of an object can be corrected. If an automatic position correction is carried out every time the position of an observation object changes, the user viewing the observation image can feel strange. Therefore, other operating modes for the position correction are also disclosed. The conditional automatic mode is a mode in which a position deviation is corrected on the condition that a deviation of the relative position exceeds an allowable range. The specified timing correction mode is a mode in which a position deviation is corrected on the condition that a command for a position correction is input by the user.

It has been observed that, in the case of such previously known techniques, the user expectation as regards the auto-configuration and the actual changes made by the surgical microscopy system may be discrepant.

### SUMMARY

Therefore, there is a need for improved techniques for automatically controlling surgical microscopy systems. In particular, there is a need for improved techniques in connection with an automatic configuration of the surgical microscopy system for imaging an object.

This object is achieved by the features of the independent claims. The features of the dependent claims define embodiments.

Aspects in connection with the automatic configuration of a surgical microscopy system having a stand and a microscope carried by the robotic stand are described below.

As a general rule, the stand may be robotic or at least partially robotic. It would also be conceivable for the stand not to be robotic.

The surgical microscopy system is configured to image a predefined object. This means that the predefined object is arranged in a particular way in the field of view of a camera, for example a microscope camera of the microscope.

The automatic configuration may comprise, for example, auto-centring on a particular reference point. The reference point may be, for example, a tip or a centre of an object. The reference point may comprise, for example, a geometric centre of gravity or a centre point or an activity centre of a plurality of objects. During such auto-centring of the field of view, the reference point is then arranged in the centre of the microscope image. For this purpose, an at least partially robotic stand is controlled accordingly.

Alternatively or additionally, the configuration may comprise setting a focus of the microscope to such a reference point (auto-focus). For example, focusing lenses may be moved such that such a reference point is placed in the focal plane.

Alternatively or additionally, it is also possible to set a particular orientation of a microscope of the surgical microscopy system in relation to such a reference point (auto-orientation): For example, a particular orientation of the microscope can be set (that is to say the optical axis can be rotationally positioned). For example, pivoting would be conceivable without the microscope being translationally moved.

A combined translational and rotational movement may also be carried out (combined auto-positioning and auto-orientation).

Alternatively or additionally, a zoom of the microscope can also be set, for example such that a particular object or a plurality of objects is/are visible in a manner filling the screen (auto-zoom).

The above techniques make it possible to carry out such an auto-configuration of the surgical microscopy system such that user expectations are met particularly well. In particular, it is possible to provide in this manner a seamless user experience which also enables a reliable system behaviour for the surgeon in high-stress situations.

For this purpose, a moving object, which forms the basis for the auto-configuration of the surgical microscopy system, is tracked during the auto-configuration of the surgical microscopy system. This means that it is checked whether the position and/or the orientation of the object change(s) in comparison with its initial position and/or orientation; this means that it is checked whether the object moves.

It is then checked whether the movement satisfies one or more criteria. For example, it is possible to check whether the position and/or orientation change(s) within a predefined spatial limit. Alternatively or additionally, it is also possible to check whether the position and/or orientation change(s) within a predefined temporal limit.

If, for example, the position changes outside the temporal limit, it is not taken into account in connection with the auto-configuration. If, for example, the orientation changes outside the temporal limit, it is not taken into account in connection with the auto-configuration. If, for example, the position changes outside the spatial limit, it is not taken into account in connection with the auto-configuration.

A computer-implemented method for controlling a surgical microscopy system is disclosed. The surgical microscopy system comprises a stand, for example a robotic or partially robotic stand. The surgical microscopy system also comprises a microscope which is carried by the stand. The microscope may have, for example, a camera for capturing a microscope image. The microscope may be, for example, a stereo microscope.

The method comprises receiving a user command. The user command requests a configuration of the surgical microscopy system for imaging a predefined object. The method also comprises initially determining a target configuration of the surgical microscopy system on the basis of (i.e. based on) the user command. The method also comprises controlling at least one component of the surgical microscopy system in order to achieve the target configuration. The method also comprises determining whether the position and/or the orientation of the movable object change(s) within a predefined temporal limit and/or within a predefined spatial limit. This determination is carried out in coordination (temporally associated) with the controlling of the at least one component. The method also comprises adapting the target configuration if the position and/or orientation of the movable object change(s) within the predefined temporal limit and/or within the predefined spatial limit.

A data processing device for controlling a surgical microscopy system is disclosed. The data processing device comprises a processor. The processor is configured to load program code from a memory and to execute it. Executing the program code causes the processor to carry out the method disclosed above for controlling the surgical microscopy system.

A surgical microscopy system having such a data processing device is also disclosed.

The features set out above and features that are described hereinbelow may be used not only in the corresponding combinations explicitly set out, but also in further combinations or in isolation, without departing from the scope of protection of the present invention.

### BRIEF DESCRIPTION OF THE FIGURES

FIG. 1 schematically illustrates a surgical microscopy system according to various examples.

FIG. 2 is a flowchart of one exemplary method.

FIG. 3 schematically illustrates auto-centring of a field of view of a camera of a surgical microscope on the tip of a surgical instrument according to various examples.

FIG. 4 corresponds to FIG. 3 and illustrates the effect of a movement of the surgical instrument in the temporal context of the auto-centring.

FIG. 5 corresponds to FIG. 4 and also illustrates a spatial limit.

FIG. 6 illustrates adapted auto-centring taking into account the spatial limit from FIG. 5.

FIG. 7 schematically illustrates a temporal limit in connection with adaptation of a target configuration during an automatic movement of a robotic stand.

FIG. 8 schematically illustrates a temporal limit in connection with adaptation of a target configuration during an automatic movement of a robotic stand.

### DETAILED DESCRIPTION

The properties, features and advantages of this invention described above and the way in which they are achieved will become clearer and more clearly understood in association with the following description of the exemplary embodiments which are explained in greater detail in association with the drawings.

The present invention is explained in greater detail below on the basis of preferred embodiments with reference to the drawings. In the figures, identical reference signs denote identical or similar elements. The figures are schematic representations of various embodiments of the invention. Elements illustrated in the figures are not necessarily illustrated as true to scale. Rather, the various elements illustrated in the figures are rendered in such a way that their function and general purpose become comprehensible to a person skilled in the art. Connections and couplings between functional units and elements illustrated in the figures may also be implemented as an indirect connection or coupling. A connection or coupling may be implemented in a wired or wireless manner. Functional units may be implemented as hardware, software or a combination of hardware and software.

Techniques in connection with the operation of surgical microscopy systems are described below. The techniques described make it possible to automatically configure one or more components of the surgical microscopy system. In this manner, the surgical microscopy system can be brought to a state in which a particular object, for example a surgical instrument, is suitably imaged. For example, it is possible to carry out auto-positioning and/or auto-centring and/or auto-orientation and/or auto-focusing and/or auto-zoom in relation to a reference point that is defined in relation to one or more objects. In particular, techniques in terms of how the surgical microscopy system can be automatically configured to image a movable object are disclosed.

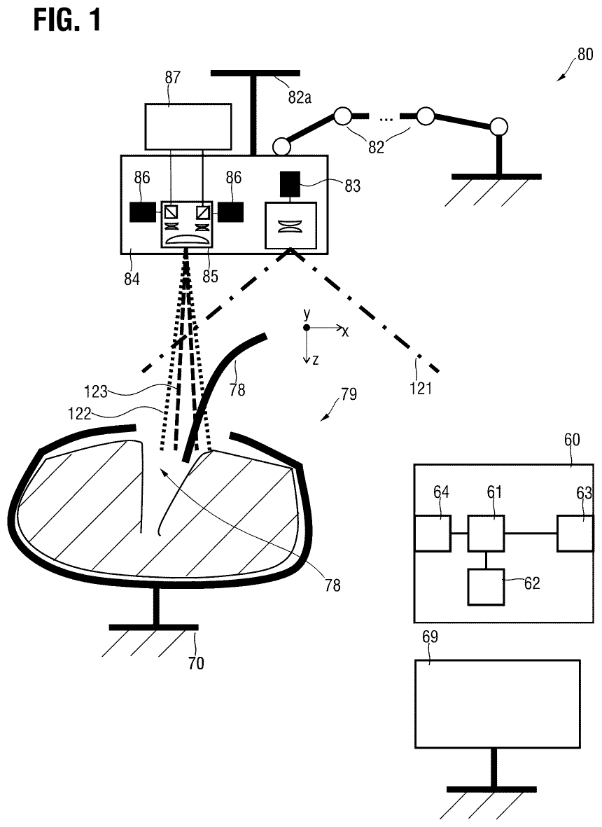

FIG. 1 schematically illustrates aspects with respect to an exemplary surgical microscopy system 80. The surgical microscopy system 80 is used to microscopically image an examination area during a surgical intervention. For this purpose, a patient 79 is placed on an operating table 70. The situs 78 with a surgical instrument 78 is shown.

The surgical microscopy system 80 comprises a robotic stand 82 which carries a positionable head part 81. Depending on the variant, the robotic stand 82 may have different degrees of freedom. Robotic stands 82 having six degrees of freedom for positioning the head part 81, that is to say translation along each of the x-axis, y-axis and z-axis and rotation about each of the x-axis, y-axis and z-axis, are known. The robotic stand 82 may, as shown in FIG. 1, have a handle 82a.

Not all variants require the stand 82 to be robotic. It would also be conceivable for the stand to be able to be operated only manually.

The head part 81 comprises a microscope 84 having optical components 85, for example an illumination optical unit, an objective optical unit, zoom optical unit, etc. (only schematically illustrated in FIG. 1).

In the example illustrated, the microscope 84 also comprises a microscope camera 86 (here a stereo camera with two channels; however, a mono optical unit would also be conceivable), with which images of the examination area can be captured and can be reproduced, for example, on a screen 69. The microscope 84 is therefore also referred to as a digital microscope. A field of view 123 of the microscope camera 86 is also shown.

In the example in FIG. 1, the microscope 84 also comprises an eyepiece 87 with an associated field of view 122. The eyepiece 87 is optional. For example, the detection beam path can be split using a beam splitter (indicated in FIG. 1), with the result that both an image can be captured by the camera 86 and observation through the eyepiece 87 is possible. Not all variants require the microscope 84 to have an eyepiece. Purely digital microscopes 84 that do not have an eyepiece are also possible.

In the example in FIG. 1, the head part 81 of the surgical microscopy system 80 that is carried by the stand 82 also comprises an environment camera 83. The environment camera 83 is optional. Whereas the environment camera 83 in the example in FIG. 1 is illustrated as being integrated in the microscope 84, it would be possible for it to be arranged separately from the microscope 84. The environment camera may be a CCD camera, for example. The environment camera may also have a depth resolution. As an alternative or in addition to the environment camera, it would also be conceivable for other assistive sensors to be present, for example a distance sensor (for instance a time-of-flight camera or an ultrasonic sensor or a sensor with structured illumination). A field of view 121 of the environment camera 83 is shown in FIG. 1.

The surgeon therefore has a plurality of options for viewing the examination area: using the eyepiece 87, using the microscope image recorded by the camera 86, or using an overview image recorded by an environment camera 83. The surgeon can also view the examination area directly (without magnification).

The various components of the surgical microscopy system 80, for example the robotic stand 82, the microscope 84 or the one or more further components, for example the environment camera 83, are controlled by a processor 61 of a data processing device 60.

The processor 61 may be, for example, in the form of a general central processor (CPU) and/or a field-programmable logic module (FPGA) and/or an application-specific integrated circuit (ASIC). The processor 61 can load program code from a memory 62 and execute it.

The processor 61 can communicate with various components of the surgical microscopy system 80 via a communication interface 64. For example, the processor 61 can control the stand 82 in order to move the head part 81 in relation to the operating table 70, for example in a translational and/or rotational manner. The processor 71 may control, for example, optical components 85 of the microscope 84 in order to implement zoom and/or auto-focus. Images from the environment camera could be read out and evaluated, if available.

The processor may evaluate a sequence of images captured by means of a camera of the surgical microscopy system in order to track an object. For this purpose, it is possible to use, for example, machine-learned algorithms which detect or optionally locate a corresponding object in the various images. Alternatively or additionally, it would be conceivable to determine the optical flow on the basis of a sequence of images; areas in which the optical flow assumes large values are areas in which a moving object is more likely to be located. Corresponding techniques are known, in principle, in the prior art (see, for example, DE 10 2021 101 694 A1); the specific algorithmic implementation used to track an object is not decisive for the techniques described herein.

The data processing device 60 also comprises a user interface 63. Commands from a surgeon or generally from a user of the surgical microscopy system 80 can be received via the user interface 63. The user interface 63 may have various configurations. For example, the user interface 63 may comprise one or more of the following components: Handles on the head part 81; foot switches; voice input; input via a graphical user interface; etc. It would be possible for the user interface 63 to provide graphical interaction via menus and buttons on the monitor 69.

Techniques in terms of how the surgical microscopy system 80 can be used to enable an automatic configuration for imaging a predefined object—for example a surgical instrument—are described below. Such a configuration may comprise, for example, aligning the microscope with the surgical instrument. For example, it is possible to carry out a lateral alignment with a surgical instrument, that is to say in the X direction and/or Y direction. The alignment may comprise, for example, centring with respect to a reference point of the object. For example, centring on a tip of a surgical instrument could be triggered. As an alternative or in addition to such a lateral alignment with an object, it is also possible to set the focus in relation to the object. For example, an objective optical unit of the microscope 84 can be controlled in order to move a lens element. It is also possible to use microscopes with a fixed focal length: in such a case, the Z position of the microscope 84 can be changed by means of the robotic stand 82. Alternatively or additionally, it is also possible to tilt the head part 81 and therefore the microscope 84 and in particular a central axis of the optical components 85 in order to image a particular object (auto-orientation). Pivoting is possible. Such a technique may have the advantage that a particular pose of the microscope 84 with respect to the object is enabled, that is to say a particular alignment and orientation. In this manner, for example, deep channels, which occur during a surgical intervention (cf. situs 78 in FIG. 1), can be imaged. Generally, such an automatic configuration for imaging a predefined object can be referred to as auto-XYZ configuration; this designation indicates that the XY extent and Z configuration of the captured image are set. Auto-XYZ can denote, for example, auto-positioning (in particular auto-centring) and/or auto-focusing and/or auto-orientation and/or auto-zoom.

For the purposes of the auto-XYZ configuration, a target configuration of one or more components of the surgical microscopy system 80 is determined and the one or more components are then controlled in order to be changed from the actual configuration to the target configuration.

In various examples, the auto-XYZ configuration is triggered by a user command. Scenarios have been observed in which the user command is received and the scene then changes. This means that one or more objects are moved and then have a different position and/or orientation than at the time at which the user command was triggered. The user command was therefore triggered or emitted in a first state of the scene, while the scene then assumes a second state. This may then result in an undesirable system behaviour in reference implementations because the auto-centring is carried out on the old or new position of the object, for example, but the user expects exactly the opposite. In other words, this means that, on account of the change in the underlying scene, the system behaviour and user expectation become discrepant.

Techniques that make it possible to better meet the user expectation than in reference implementations in connection with an auto-XYZ configuration triggered by a user command are described below. A deterministic system behaviour is enabled and provides reproducible and comprehensible results in very different situations and/or in view of very different and also dynamic scenes. Such techniques are based on the knowledge that, in particular in high-stress situations under time pressure, as typically occur in a surgical environment, it is necessary for the system behaviour of an automatic controller to exactly match the user expectation.

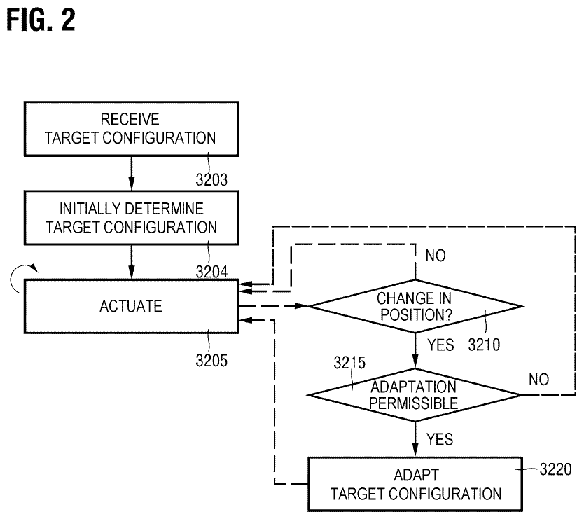

FIG. 2 is a flowchart of one exemplary method. The method in FIG. 2 relates to techniques for carrying out an assistance functionality. The assistance functionality comprises or consists of an auto-XYZ configuration of the surgical microscopy system.

In particular, FIG. 2 relates to techniques for carrying out the assistance functionality in relation to moving objects. Examples of moving objects are, in particular, surgical instruments which are handled by a surgeon.

Examples of surgical instruments are generally: a scalpel, forceps, scissors, a needle holder, a clamp, an aspirator, a trocar, a coagulator, an electrocauter, a retractor, a drill, a spreader, an osteotome, suture material, a knot pusher, a periosteal elevator, a haemostat, a lancet, drainage, a thread cutter, a spatula, ultrasonic aspirators.

A user command is received in box 3203. The user command implicitly or explicitly requests, for example, the configuration of the microscope camera for imaging a predefined object, for example a surgical instrument. The user command may request a particular assistance functionality. The user command is received by a user interface (cf. FIG. 1). The user command may request, for example, auto-alignment or auto-focusing. The user command may trigger an auto-XYZ configuration.

A target configuration of the surgical microscopy system is initially determined in box 3204. This target configuration is determined on the basis of a user command from box 3203. The target configuration may be determined for the scene with one or more objects that is currently imaged by means of a camera of the surgical microscopy system. This target configuration relates to one or more components of the surgical microscopy system. For example, the target configuration may indicate how the robotic stand is intended to arrange and/or orient the head part. One or more settings could also be indicated for the microscope, for example a magnification of a zoom lens optical system to be used or a setting for the auto-focus and/or filters to be used etc.

At least one component of the surgical microscopy system is then controlled in box 3205 in order to achieve the target configuration. Depending on implementation variants, the controlling in box 3205 may assume different forms. For example, box 3205 could comprise transferring a setting value to control hardware of the surgical microscopy system. The control hardware may then convert this setting value into a sequence of specific control commands, for example analogue values. In this manner, an actual configuration of the surgical microscopy system is gradually changed into the target configuration. However, it would also be conceivable for a sequence of appropriate setting values to be determined in box 3205 and to be directly transferred to the corresponding one or more components.

Depending on the assistance functionality, different components can be controlled in box 3205. For example, the field of view of the microscope could be automatically centred on a predefined object. Such centring can be carried out in two dimensions, that is to say using an XY translation of the head part. In such a situation, the robotic stand can be controlled in order to implement such a translation. Alternatively or additionally, it would also be conceivable for automatic centring to be carried out in six dimensions, that is to say using rotation and translation of the head part, in order to look into a situs etc., for example. Alternatively or additionally, auto-focusing on a particular predefined object could also be carried out. In such a case, it is possible for a movable optical unit of an objective of the microscope to be controlled. However, in such an example, it would also be possible—in particular if the optical components are equipped with a fixed focal length—to carry out Z-positioning of the head part. In a further scenario, it would be conceivable to set the field of view for a set of objects (for example those objects which are recognized as relevant) by adapting a zoom factor. For example, the zoom factor could be selected to be as large as possible, with the result that all selected objects are still visible (there is then no need for a robotic stand because only the optical unit is moved). Some objects can then be arranged close to the edge of the zoomed field of view.

The controlling in box 3205 causes the one or more components of the surgical microscopy system to be adjusted (in order to achieve the target configuration). The adjustment of the one or more components is not carried out instantaneously, but rather requires a certain period of time (that is to say has a certain latency). The reason for this is, for example, the limitation of actuators, for example servomotors (acceleration, maximum speed). Further reasons are the mass inertia and the necessary positioning accuracy that can cause a slow adjustment. For example, it may also be necessary in the case of exposed components—in particular the robotic stand which carries the head part and moves the head part in the operating theatre—to limit the speed of the movement in order to avoid endangering the surrounding personnel as a result of collision. Typical periods for moving the robotic stand into a particular position are on the timescale of several seconds to several tens of seconds. The auto-focusing by adjusting lens elements of an objective also requires a certain time, typically up to 1 second or 2 seconds. The same applies to a zoom functionality.

Various examples are based on the knowledge that, on account of such latency in the implementation of a particular configuration of the surgical microscopy system, it may also happen that the scene on which the target configuration from box 3204 is based also changes during the setting period (or even shortly after setting has been completed). This specifically means that the moving object may move away from that reference position (for example in the centre of the field of view of the camera) which was used as the basis for determining the target configuration. The target configuration from box 3204 is therefore outdated. In general terms, this is due to the fact that the timescales on which, on the one hand, the moving objects move and on which, on the other hand, one or more components of the surgical microscopy system are adjusted in order to achieve the target configuration are comparable.

Accordingly, box 3210 checks whether the position of the movable object changes. This can be carried out, for example, during or in coordination with the controlling in box 3205.

For example, a particular tolerance can be accepted during the check in box 3210, with the result that slight movements are ignored (high-frequency readjustment is thus avoided).

If no (significant) movement is identified in box 3210, the target configuration on which the controlling in box 3205 is based is not adapted (“no” path from box 3210).

If the position of the movable object has changed (significantly) (“yes” path from box 3210), box 3215 is carried out. Box 3215 determines whether the position and/or orientation of the movable object has/have changed within a predefined temporal limit and/or spatial limit. It is thus checked whether adaptation of the target configuration is permissible because the position and/or orientation of the movable object is/are changed only within the temporal limit and/or spatial limit. Particularly large changes and/or changes that are not in a particularly close temporal context with the controlling in box 3205 can therefore be avoided. On the other hand, some movements which are in a sufficiently close spatial and/or temporal context are taken into account during controlling. If the adaptation is permissible, the target configuration is adapted in box 3220 and the controlling in box 3205 then takes into account the adapted target configuration.

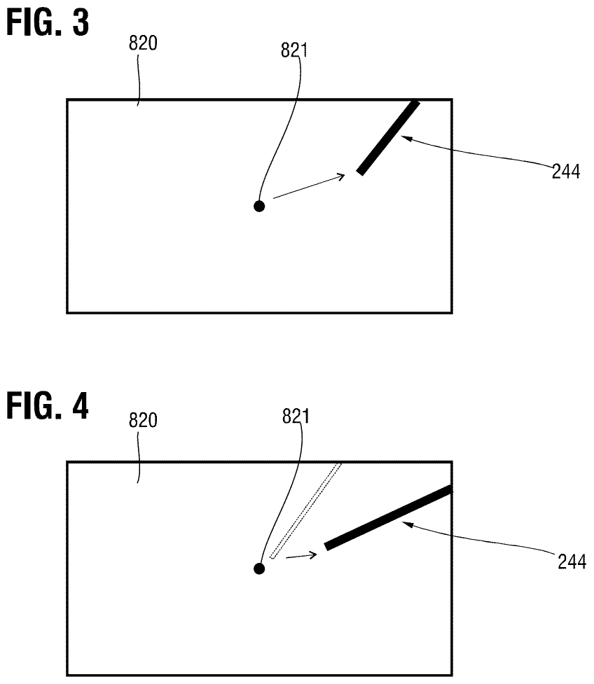

The effects of such a method are discussed next on the basis of a specific example. This is carried out separately from reference implementations. FIG. 3 shows the initial state. A surgical instrument 244 is arranged with an offset with respect to the centre 821 of the microscope image 820. An auto-centring command is then received (lateral XY centring); the command requests the tip of the surgical instrument 244 to be arranged in the centre. This is illustrated by the arrow in FIG. 3. While the head part is moved in order to implement this auto-centring, the surgical instrument 244 is also moved, cf. the arrow in FIG. 4. This therefore means that the scene on which the auto-centring is based also changes during the movement of the head part. This causes (without further measures for adapting the target configuration, that is to say according to reference implementations, for example the “specified timing correction mode” described in U.S. Pat. No. 10,456,035 B2) the centre 126 to not coincide with the tip of the surgical instrument 244 after the end of the positioning process. This may result, at least under certain conditions, in the user expectation (for instance: “centred on the tool tip”) not being met. In the example in FIG. 3 and FIG. 4, one-off centring is therefore carried out: after activating the assistive function (for example via a voice command or a button of a foot control panel), the head part is centred on the surgical instrument activity present at the time of activation. Since the surgeon generally continues his work while the surgical microscope is still carrying out its movement, this results in the centre of the field of view 123 of the microscope 84 not corresponding to the surgical instrument position after the movement of the surgical microscope. For correct centring, the one-off centring would have to be activated again and the surgical instrument activity would have to be paused. However, this is disadvantageous since it results in an interruption in the surgery workflow and possibly the surgeon's concentration. Accordingly, it would also be conceivable, in another (disadvantageous) reference scenario (for example the “conditional automatic mode” described in U.S. Pat. No. 10,456,035 B2), for the movement of the surgical instrument 244 to be continuously followed. Therefore, there is continuous centring on the activity of the surgical instrument, that is to say, after the assistive function has been activated (for example via a voice command or a button of a foot control panel), the microscope is continuously centred on the activity until the assistive function is deactivated again. However, this reference implementation is often considered to be disruptive since it is not possible to distinguish between desired and undesired movements of the objects and the surgical microscope would consequently carry out movements which would disrupt the surgeon's concentration and focus (and consequently would also increase the risk to the patient).

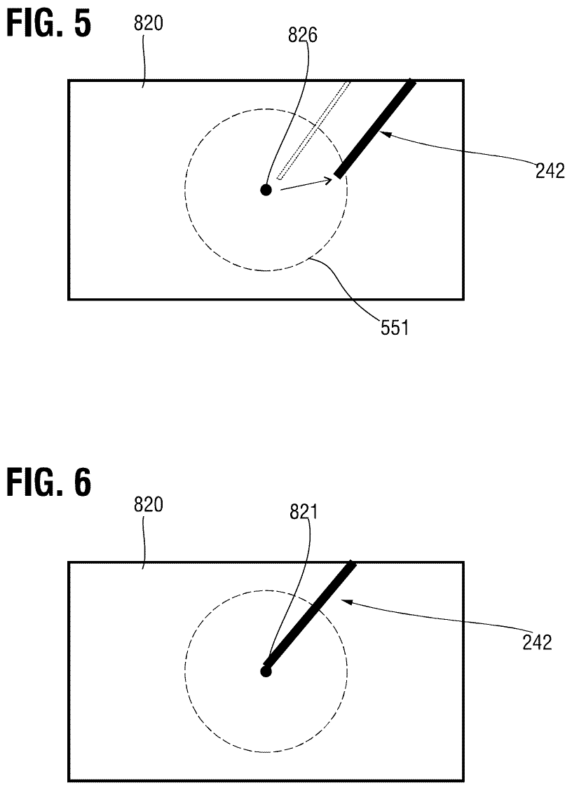

Such problems of the reference scenarios described above are at least alleviated by taking into account the temporal and/or spatial limit in box 3215. Movements of the object result in adaptation of the target configuration only within the temporal limit and/or spatial limit. That is shown in FIG. 5.

FIG. 5 illustrates aspects in relation to a spatial limit 551. In the example illustrated, the spatial limit is determined in connection with the target configuration initially determined on the basis of the user command (cf. box 3204). In detail: The predefined object, here the tip of the surgical instrument, is intended to be positioned in the centre of the field of view 123 of the microscope camera and therefore in the centre of the microscope image using the initial target configuration. This defines a reference position of the predefined object; in the present example, this reference position is in the centre of the microscope image. The spatial limit is defined as the maximum distance in relation to a reference position associated with the initial target configuration.

In the example in FIG. 5, the tip 242 moves away (illustrated by the arrow) from this reference position, specifically by a distance which is less than the spatial limit 551. Therefore, the target configuration (initially determined on the basis of the user command) is adapted, such that the centre of the field of view follows the moving surgical instrument 242 and the moving tip 242 is ultimately arranged in the centre of the microscope image 820, compare FIG. 6. If the movement of the tip 242 were greater than the spatial limit, it would be possible to output an error and/or to terminate the auto-centring (“no” path from box 3125 in FIG. 2).

A scenario in which the spatial limit 551 is determined on the basis of a reference position of the object that is associated with the target configuration initially determined on the basis of the user command was described above. It would also be conceivable for the spatial limit 551 to be adapted again after the surgical instrument 242 has been repositioned within the spatial limit 551. The spatial limit 551 can therefore be “tightened up”. This means that the reference position can be adapted in accordance with the correspondingly adapted target configuration. Such iterative adaptation of the spatial limit 551 can be continued until the actual position and the target position of the surgical instrument 242 stably converge. This corresponds, for instance, to the function of a low-pass filter: smaller movements of the surgical instrument 242 cause “tightening-up” of the auto-centring, whereas larger movements of the surgical instrument 242 do not cause any adaptation of the auto-centring.

A spatial limit, as discussed in FIG. 5 and FIG. 6, can be combined with a temporal limit. However, the temporal limit can also be used without a spatial limit. Aspects with the temporal limit are discussed next.

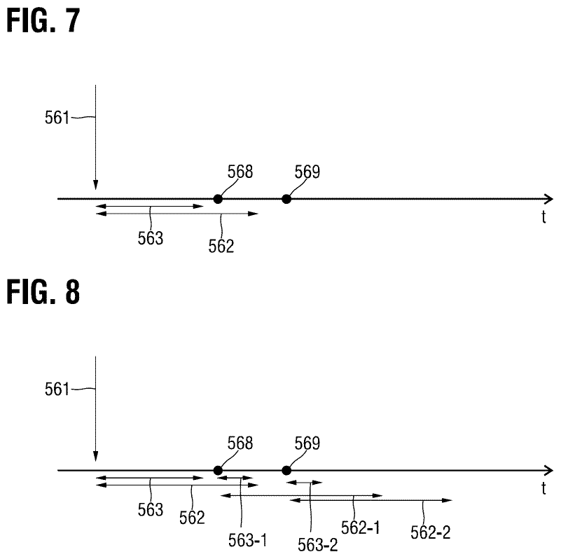

FIG. 7 illustrates aspects in relation to a temporal limit 562. The temporal limit 562 indicates a period of time within which movements of the predefined object, which forms the basis for the target configuration of the surgical microscopy system, are potentially taken into account in order to adapt the target configuration. If a movement therefore takes place within the temporal limit, it is taken into account (for example provided that the movement is also within the spatial limit) and is otherwise not taken into account.

In FIG. 7, a user command 561 is received (which requests, for example, auto-centring, so that the target configuration can be initially determined, cf. box 3204) and one or more components of the surgical microscopy system are then adjusted in a period of time 563. This period of time may be dependent, for example, on a trajectory travelled by the robotic stand in order to suitably position the head part.

The temporal limit 562 has, in the example in FIG. 7, a length that is 150% of the period of time 563. That is to say, the temporal limit 563 is determined by the period for achieving the target configuration initially determined on the basis of the user command 561.

The object is moved 568 within the temporal limit 562; for example, the surgical instrument is moved; this movement is taken into account in order to adapt the target configuration because it takes place within the temporal limit. However, a later movement 569 is outside the temporal limit 562 and therefore is not taken into account in order to adapt the target configuration.

In the example in FIG. 7, the temporal limit 562 is initially defined, specifically by the duration of the period 563, which is determined on the basis of the trajectory initially determined on the basis of the user command for achieving the target configuration. However, it would also be possible for the temporal limit to be dynamically adapted, which is shown in FIG. 8.

FIG. 8 illustrates aspects in relation to a repeatedly adapted temporal limit 562, 562-1, 562-2. The limit 562, 562-1, 562-2 is adapted (that is to say “tightened up”) until the actual configuration stably corresponds to the target configuration, that is to say until the target configuration is finally achieved. In the example in FIG. 7, the movement 568 (as discussed in connection with FIG. 7) triggers adaptation of the target configuration (the target position has not yet been finally reached at this time); the robotic stand is therefore controlled again, for example in order to re-centre the field of view; the robotic stand is controlled during the period of time 563-1. The renewed movement 568, in the example in FIG. 8, triggers a reinitialization of the temporal limit 562-1, with the result that the movement 569 is now also taken into account. The movement 569 again triggers a movement (period of time 563-2), which in turn triggers a reinitialization of the temporal limit 562-2. The actual configuration is then stably the same as the target configuration and there is no longer any reinitialization of the temporal limit. The object is then finally and stably arranged in the centre of the microscope image, for example.

Whereas the scenarios in FIG. 7 and FIG. 8 show examples in which the temporal limit 562 in each case has a starting time that coincides with the starting time for setting one or more components in the period of time 563, it would also be generally conceivable for the temporal limits described herein to have a different starting time. For example, the beginning of a temporal limit could be offset by a particular predefined period in comparison with the reception of the user command 561 and/or in comparison with the beginning of the movement of one or more components of the surgical microscopy system. In addition to such a temporal shift, it would also be possible to start the time limit in an event-controlled manner. For example, it could be checked whether the microscope has been moved by a certain distance.

In connection with the dimensioning of the temporal and/or spatial limits: It would be possible for their magnitudes to be firmly predefined. However, it would also be possible for their magnitudes to be dynamically determined, for example on the basis of a type of object; a setting of one or more components of the surgical microscopy system, for example magnification of the microscope, working distance, surgical context (for example surgery type), position of the patient, etc. Such techniques are based on the knowledge that certain object types typically have a smaller movement than other object types. This means that the tolerance in connection with the adaptation of the target configuration can be selected to be smaller for such object types than for other object types.

Various scenarios can therefore be implemented using the spatial limit and/or the temporal limit. Some exemplary scenarios are discussed below. In a first scenario, the surgeon activates the auto-centring on a particular surgical instrument once. The position and/or orientation of the microscope is/are then calculated on the basis of the current positioning of the surgical instrument in relation to the microscope, that is to say the target configuration for the robotic stand is initially determined. In this context, an area or a volume around this target position or around the tip of the surgical instrument, within which the target pose can be changed during travel, is also defined. This therefore means that the spatial limit is determined in relation to a reference position determined on the basis of the initial target configuration. The robotic movement into the target configuration then begins. The activity of the surgical instrument is continuously evaluated (tracking). The target pose is adapted as long as the tip of the surgical instrument is within the previously calculated area or the previously calculated volume. Upon reaching the target pose, the robotic movement is ended and there is also no further continuous adaptation of the target configuration. This therefore corresponds to a spatial limit in combination with a temporal limit that is defined by actually reaching the target position. In a further scenario, the one-off centring by the surgeon, as already explained above, is activated. A target configuration—for example a target pose of the robotic stand—is then determined. A time, within which the movement of the surgical instrument results in adaptation of the target configuration, is also determined (for example on the basis of the estimated travel time for achieving the target configuration). The robotic movement is then started and the activity of the surgical instrument is continuously evaluated (tracking). The target configuration is adapted during the previously determined temporal limit on the basis of the detected activity. Combinations of these two variants are also conceivable.

The features set out above and features that are described hereinbelow may be used not only in the corresponding combinations explicitly set out, but also in further combinations or in isolation, without departing from the scope of protection of the present invention.

For example, various aspects in connection with an auto-XYZ configuration in relation to a surgical instrument were described above. However, as a general rule, it would be conceivable for an auto-XYZ configuration to also be carried out on other objects, for example characteristic anatomical features of the patient.

Furthermore, aspects in which an auto-XYZ configuration is carried out in relation to a single object were described above. However, it would generally be conceivable for the auto-XYZ configuration to be carried out in relation to a plurality of objects. For example, a reference point dependent on the positions of the plurality of objects (for example a geometric centre point or an activity centre) can be determined and an auto-XYZ configuration can then be carried out in relation to this reference point. For example, a subset of relevant objects could be selected from all visible objects, wherein the auto-XYZ configuration is then carried out in relation to the at least one object in this subset.

Furthermore, various aspects in connection with a robotic stand were described above. It is not absolutely necessary for the surgical microscopy system to have a robotic stand. The surgical microscopy system could also have a partially robotic stand or a manual stand.

1. A computer-implemented method for controlling a surgical microscopy system having a stand and a microscope carried by the stand,
wherein the method comprises:
receiving a user command that requests a configuration of the surgical microscopy system for imaging a predefined object,
initially determining a target configuration of the surgical microscopy system based on the user command,
controlling at least one component of the surgical microscopy system in order to achieve the target configuration,
temporally related with the controlling of the at least one component: determining whether the position and/or orientation of the movable object change(s) within a predefined temporal limit and/or within a predefined spatial limit, and
if the position and/or orientation of the movable object change(s) within the predefined temporal limit and/or within the predefined spatial limit, adapting the target configuration.

2. The computer-implemented method according to claim 1, wherein the method furthermore comprises:
determining a reference position of the predefined object based on the target configuration initially determined based on the user command,
wherein the spatial limit is determined in relation to the reference position.

3. The computer-implemented method according to claim 2, wherein the method furthermore comprises:
adapting the reference position based on the adapted target configuration if the position of the movable object changes within the predefined temporal limit and/or the predefined spatial limit.

4. The computer-implemented method according to claim 1, wherein the method furthermore comprises:
initially determining the temporal limit based on a period of time for achieving the target configuration initially determined based on the user command.

5. The computer-implemented method according to claim 4, wherein the method furthermore comprises:
adapting the temporal limit initially determined based on the period of time until the target configuration is finally achieved.

6. The computer-implemented method according to claim 1, wherein the method furthermore comprises:
determining a type of movable object,
wherein the predefined temporal and/or spatial limit depends on the type of predefined object.

7. The computer-implemented method according to claim 1,
wherein the user command requests auto-centring of a field of view of the microscope on the predefined object,
wherein the at least one component comprises the at least partially robotic stand.

8. The computer-implemented method according to claim 1,
wherein the user command requests auto-focusing of an image plane of the microscope on the predefined object,
wherein the at least one component comprises an objective optical unit of the microscope and/or the at least partially robotic stand.

9. A data processing device for controlling a surgical microscopy system, wherein the data processing device comprises a processor which is configured to load program code from a memory and to execute it, wherein the execution of the program code causes the processor to carry out the following steps:
receiving a user command that requests a configuration of the surgical microscopy system for imaging a predefined object,
initially determining a target configuration of the surgical microscopy system based on the user command,
controlling at least one component of the surgical microscopy system in order to achieve the target configuration,
temporally related with the controlling of the at least one component: determining whether the position and/or orientation of the movable object change(s) within a predefined temporal limit and/or within a predefined spatial limit, and
if the position and/or orientation of the movable object change(s) within the predefined temporal limit and/or within the predefined spatial limit, adapting the target configuration.

10. (canceled)

11. A surgical microscopy system which comprises the data processing device according to claim 9.
